(19) United States
(12) Patent Application Publication
(10) Pub. No.: US 2006/0122567 A1
(43) Pub. Date: Jun. 8, 2006

(54) Dispenser

(76) Inventor: Patrick Kennedy, Breckenridge, TX (US)

Correspondence Address: JACKIE JAY SCHWARTZ, 1350 Broadway, Suite 1510, NEW YORK, NY, 10018, US

(21) Appl. No.: 11/272,045

(22) Filed: Nov. 10, 2005

Related U.S. Application Data
(60) Provisional application No. 60/627,631, filed on Nov. 12, 2004.

(51) Int. Cl. A61M 35/00

(52) U.S. Cl. 604/289

(57) ABSTRACT

The dispenser includes a housing having a first closed end, and a wall extending from a periphery thereof forming a reservoir for retaining a dry lightweight substance. An applicator releasably, rotatably connects to and seals an end of the reservoir opposite the first closed end so as to seal the substance within the reservoir for selectively retaining the dry lightweight substance within the reservoir. The applicator is partially recessed within the reservoir and includes a soft absorbent material extending therearound. Upon the apparatus being moved such that the substance contacts the soft absorbent material, an amount of the substance is caused to be retained by the soft absorbent material. The applicator is placed against a surface and the apparatus is moved along the surface causing the applicator to rotate thereby applying the substance retained by the soft absorbent material to the surface.

### CROSS REFERENCE TO RELATED APPLICATIONS

This is a Non-Provisional application which claims priority from U.S. Provisional Application 60/627,631 filed Nov. 12, 2004.

### FIELD OF THE INVENTION

The present invention relates generally to particulate matter dispensers, and more specifically, to a dispenser for minimizing an amount of particulate matter becoming airborne upon use thereof.

### DESCRIPTION OF THE PRIOR ART

Numerous other methods of and apparatus for dispensing powder and/or dry light material (hereinafter referred to as powder) exist in the prior art. An exemplary container for dispensing of a powder substance includes a top having perforations extending therethrough. The top is rotatable and when turned slightly is able to obstruct the perforations and prevent dispensing. These containers, (hereinafter referred to as “shaker cans”) when shaken into hands for rubbing the powder onto an area of the skin or when shaken directly onto the skin, allow large amounts of powder to escape into the air. The airborne powder causes irritation to the eyes and lungs of humans and/or animals and may even contain carcinogens or other harmful ingredients or elements. Other applicator apparatus require the addition of a flow agent to be mixed with the particulate matter in order to be dispensed therefrom via a rolling ball.

Many fine powders found in cosmetics which are used for face and body applications may be serious irritants to human and/or animal lungs and eyes. The wide spread use of “shaker cans” has ignored the scientific community's warnings of potentially dangerous side effects from airborne particulates. Talc is commonly used after bathing or to soothe irritated or inflamed skin. However, what is not commonly known is the chemical similarity of talc to the known carcinogen asbestos. In fact, talc and asbestos are virtually chemically identical. Therefore, it is desirable to reduce the amount of talc that contacts any mucous membranes of either humans or animals.

A 1993 US National Toxicology Program report found that cosmetic grade talc, even though it contained no asbestos-like fibers, caused tumors in animals. It would therefore appear that, whether or not asbestos-like fibers are present, cosmetic grade talcum powder is a carcinogen. Ref: National toxicology program. “Toxicology and carcinogenesis studies of talc (GAS No 14807-96-6) in F344/N rats and B6C3F mice (inhalation studies)”. Technical report Series No 421, September 1993.

The design of current product particulate matter dispensers do not consider the safety of the user. Thus, it is desirable to produce a dispenser that is capable of minimizing the airborne particulates thereby reducing unwanted irritation due to the airborne dispensed particulate matter.

A system according to invention principles address these deficiencies and associated problems.

### SUMMARY OF THE PRESENT INVENTION

The dispenser includes a housing having a first closed end, and a wall extending from a periphery thereof forming a reservoir for retaining a dry lightweight substance. An applicator releasably, rotatably connects to and seals an end of the reservoir opposite the first closed end so as to seal the substance within the reservoir for selectively retaining the dry lightweight substance within the reservoir. The applicator is partially recessed within the reservoir and includes a soft absorbent material extending therearound. Upon the apparatus being moved such that the substance contacts the soft absorbent material, an amount of the substance is caused to be retained by the soft absorbent material. The applicator is placed against a surface and the apparatus is moved along the surface causing the applicator to rotate thereby applying the substance retained by the soft absorbent material to the surface.

The dispenser includes a housing for retaining powders or particulate matter therein. An applicator is removeably connected at an open end of the housing for applying to a surface the at least one of powder or particulate matter therein. The dispenser minimizes the release of powders or particulate matter into the air and reduces the irritation of the lungs and/or eyes of the user that results from the at least one of powder and particulate matter. The dispenser eliminates the need to shake and cause to become airborne, the particulate matter retained in the housing in order to effectively apply and/or dispense the particulate matter to a surface. The dispenser is advantageously refillable and re-useable.

### BRIEF DESCRIPTION OF THE DRAWING FIGURES

FIG. 1 is a cross-sectional view of the powder dispenser according to invention principles taken through a length side of the applicator;

FIG. 2 is a cross-sectional view of the powder dispenser according to invention principles taken through a width side of the applicator;

FIG. 3 is a side cross-sectional view of the powder dispenser according to invention principles showing movement of the applicator to provide access to the reservoir; and

FIG. 4 is a perspective view of the powder dispenser according to invention principles.

### DETAILED DESCRIPTION OF THE INVENTION

The powder dispenser 10 dispenses powder directly onto skin or other surfaces. Powder is dispensed from a storage container without having to shake the storage container as is typically associated with prior art powder dispensers whereby powder is shaken into a user's hands for an application. By eliminating the need to shake the powder dispenser, the dispenser 10 of the present invention minimizes the amount of particulates which become airborne.

The dispenser includes a housing having a reservoir for retaining particulate matter therein such as dry lightweight material. The material retained in the reservoir is dispensed via an applicator connected at an open end of the housing. The applicator includes a cylinder that is at least one of formed from or coated with an absorbent material that is rotatable about an axis at the open end of the housing. The absorbent material may be at least one of a natural and synthetic material. The natural absorbent material is at least one of sheepskin, sponge and cotton and the synthetic absorbent material is at least one of nylon, acrylic, and porous (dry form) foam. However, any absorbent material able to releaseably hold particulate matter and apply the particulate matter directly to a surface upon contact may be used. The applicator is removeably connected to the storage container for easy cleaning and/or replacement thereof. The connection between the applicator and the housing forms a hermetic seal insuring that the particulate matter retained within the housing is not contaminated by airborne pathogens or toxicants. The seal further insures that the particulate matter within the housing is not expelled undesirably therefrom. The dispenser operates using a gravity feed method, whereby, when the housing is inverted the material in the reservoir is caused to contact the absorbent material and be temporarily retained thereby. When the applicator is placed against a surface and caused to rotate, the material is temporarily retained by the absorbent material and applied to the contacted surface. The rotation of the cylinder allows for the supply of particulate matter on the absorbent material to be continually replaced and applied to the surface.

The reduction in airborne particulate matter results in reduced irritation of at least one of eyes and respiratory systems of persons within an area around where the particulate matter is applied. The dispenser includes a cylinder of soft absorbent material which is partially inset and removeably affixed to the top of a container. The dispenser also includes a protective, removable cap for covering the absorbent material when not in use. The container retains the powder and/or dry light material and may or may not be refillable.

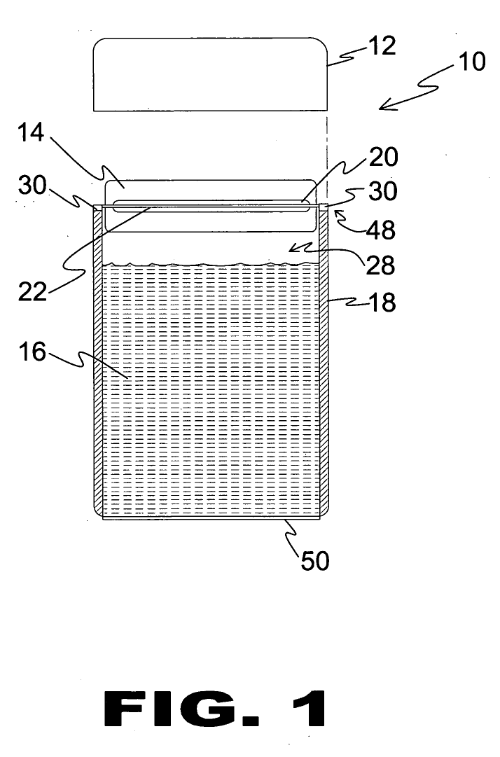

FIG. 1 is a perspective view of the powder dispenser 10. The powder dispenser 10 includes a housing 18 having a top end 48 and a bottom end 50. The housing 18 is cylindrical and has a substantially rectangular cross-section. However, the housing 18 may be formed in any geometric shape. The bottom end 50 of the housing 18 is closed so that the interior of the housing 18 forms a reservoir 28 for retaining a dry lightweight material product 16 therein. Hereinafter, all the dry lightweight material 16 will be referred to as powder 16. The top end 48 of the housing 18 is open to receive and distribute the powder 16 therethrough via an applicator 14.

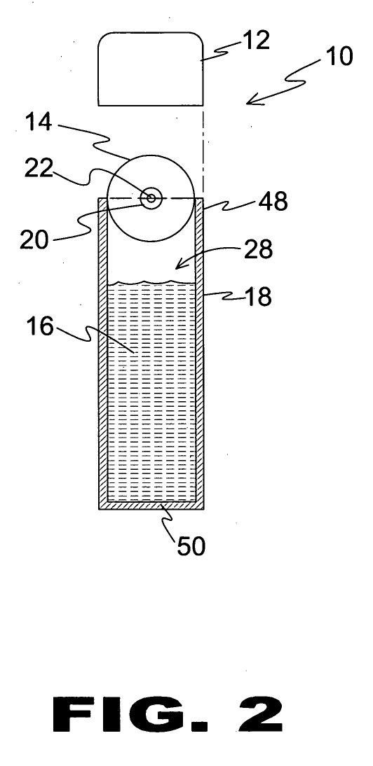

The applicator 14 is positioned to cover the open top end 48 of the housing 18 and includes a sleeve 20 extending through a central section thereof and extending along a length thereof. A pin 22 extends through the sleeve and is rotatably received a the top end 48 of the housing 18. The pin 22 will be discussed hereinafter in FIG. 2. An absorbent material 52 is removeably positioned around the sleeve 20. When the applicator 14 is positioned at the top side 48 of the housing 18, the absorbent material 52 is partially recessed within the dispenser reservoir 28 and partially exposed above the top end 48 of the housing 18. The pin 22 rotatively connects the applicator 14 to cover the top end 48 of the housing 18.

The absorbent material 52 is soft and pliable so as to be removably secured around the sleeve 20. The sleeve 20 has an internal diameter larger than the diameter of the pin 22 extending therethrough such that the sleeve 20 is able to rotate 360 degrees about the pin 22. Alternatively, the sleeve 20 and pin 22 may be formed integrally with one another. Additionally, once the sleeve 20 is surrounded by the absorbent material 52, the absorbent material 52 and sleeve 20 are rotatable 360 degrees, in tandem, about the pin 22. Preferably, the absorbent material 52 is removably secured to the sleeve 20. Alternatively, the applicator 14 maybe formed entirely from the absorbent material and the sleeve 20 may be formed integral with the absorbent material 52. The soft absorbent material 52 is at least one of a natural material including but not limited to sheepskin, sponge, natural cloth, organically produced foam and cotton or a synthetic material including but not limited to nylon, acrylic, synthetic cloth and porous (dry form) foam, plastic or chemically produced foam. Additionally, the soft absorbent material being formed from sheepskin is described for exemplary purposes only and the soft absorbent material may be any animal skin having short, tightly woven hair and able to retain powder 16 therein. The soft absorbent material 52 includes a rounded (i.e. bound) edge in order to minimize bleeding of the powder 16 retained thereby. The minimized bleeding due to the rounded edge further reduces an amount of particulate matter caused to be airborne during application thereof.

The applicator 14 is releasably connected to the housing 18. The top end 48 of the housing includes two channels 30 formed on either side thereof. The channels 30 are aligned substantially 180 degrees from one another. Each end of the pin 22 is rotatably received within a respective channel 30 in housing 18. The dimensions of the channels 30 are such that the pin 22 does not fall out when the housing 18 is inverted. Alternatively, a releasable locking mechainsim may be positioned in each channel 30 to releasably secure the pin 22 therein. The pin 22 is releasably connected within the channel 30 thereby allowing a user to at least one of replace the applicator 14, clean the applicator 14 and insert additional powder 16 within the reservoir 28. The pin 22 may be retained within the channels 30 via at least one of a snap-fit mechanism and a tension spring mechanism.

A cap 12 releasably connects to the top end 48 of the housing 18 and is positioned to obstruct access to the applicator 14 when the powder dispenser 10 is not in use. The cap 12 is shown having a rectangular cross-section. However, the cap 12 will be any shape able to be retained on the housing 18.

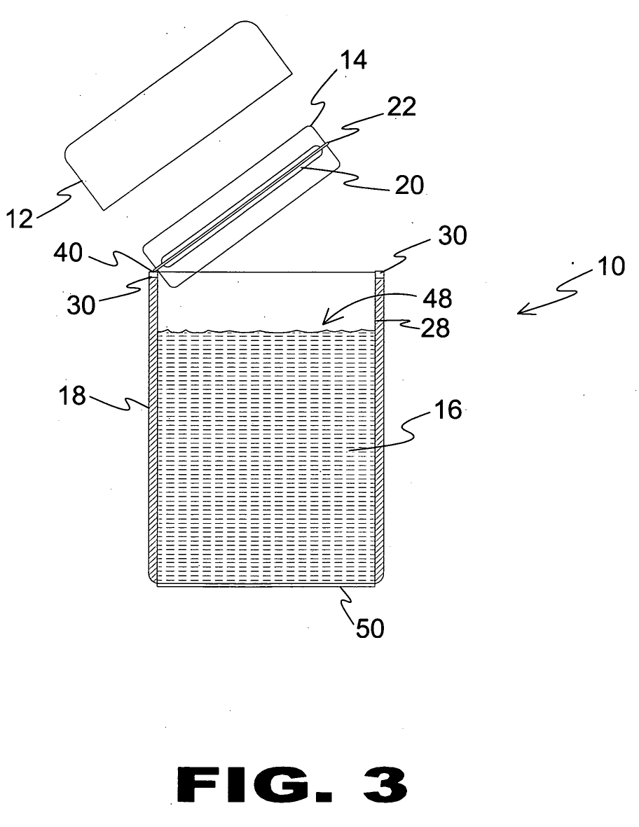

The housing 18 is grasped by a user to apply the powder 16 to a skin surface of either a human or an animal. When the top end 48 of the housing 18 is inverted, the powder 16 pools at the top of the dispenser reservoir 28 and contacts the surface of the applicator 14 resting within the dispenser reservoir 28. The user, as will be discussed hereinafter with respect to FIG. 3, positions the exposed surface of the soft absorbent material 52 of the applicator 14 (i.e. the portion of the soft absorbent material 52 not within the reservoir 28) against a surface where the powder 16 is to be applied. The powder dispenser 10 is moved along the surface causing the sleeve 20 and the absorbent material 52 to rotate around the pin 22. When the absorbent material 52 rotates, the surface within the dispenser reservoir 28 receives powder 16 from within the dispenser reservoir 28. The surface of the soft absorbent material 52 receiving the powder 16 is then rotated for applying powder 16 onto a desired area of a surface. The soft absorbent material 52 allows for uniform distribution of the powder 16 while simultaneously minimizing the amount of powder 16 becoming airborne during application thereof.

The housing 18 of the dispenser 10 may be formed from at least one of stainless steel or other metal and injection molded plastic material. It is desirable for the container to have a rectangular shape to range between one inch and five inches in diameter and ranging between one-half of one inch and three inches in depth and to range between two inches and ten inches in height. Alternatively, the container may be formed as a square that ranges between three inches by three inches and five inches by five inches squared and ranging between one half of one inch and two inches in depth. Furthermore, the dispenser 10 may be any other geometric shape such as circular or oval.

FIG. 2 shows a side cross-sectional view of the powder dispenser 10 of the present invention. The powder dispenser 10 includes the housing 18 having the top end 48 and the bottom end 50. Descriptive properties of the housing 18, cap 12, and applicator 14 shown herein are substantially similar to those described above with reference to FIG. 1.

A applicator 14 is positioned to cover the open top end 48 of the housing 18. The applicator 14 is partially recessed in the dispenser reservoir 28 and partially exposed above the top end 48 of the housing 18. The applicator 14 is cylindrical in shape and includes a soft absorbent material 52 positioned around a sleeve 20. The pin 22 extends through the cylindrical sleeve 20 for rotatively connecting the applicator 14 to the top end 48 of the housing 18. Alternatively, the pin 22 may be integrally formed with the sleeve 20 and applicator 14.

The user applies the powder 16 using the dispenser 10 whereby the sleeve 20 is rotated about the pin 22 causing the soft absorbent material 52 to roll along the skin of the user 24. As the applicator 14 rotates, the portion of the absorbent material 52 within the reservoir 28 receives and retains powder 16 from the dispenser reservoir 28 and, as the applicator is rotated, the portion of the absorbent material 52 which has received powder 16, is caused to contact the skin surface and deposits the powder 16 onto the desired area without introducing the powder particles to the air thereby reducing and/or eliminating potential irritants from being inhaled by the user 24.

FIG. 3 is a side cross-sectional perspective view of the powder dispenser 10 of the present invention showing the applicator 14 being removed. The powder dispenser 10 includes the housing 18 for retaining the powder 16 in the dispenser reservoir 28. The housing 18 includes the top end 48 and the bottom end 50. Descriptive properties of the housing 18, cap 12, and applicator 14 shown herein are substantially similar to those described above with reference to FIG. 1.

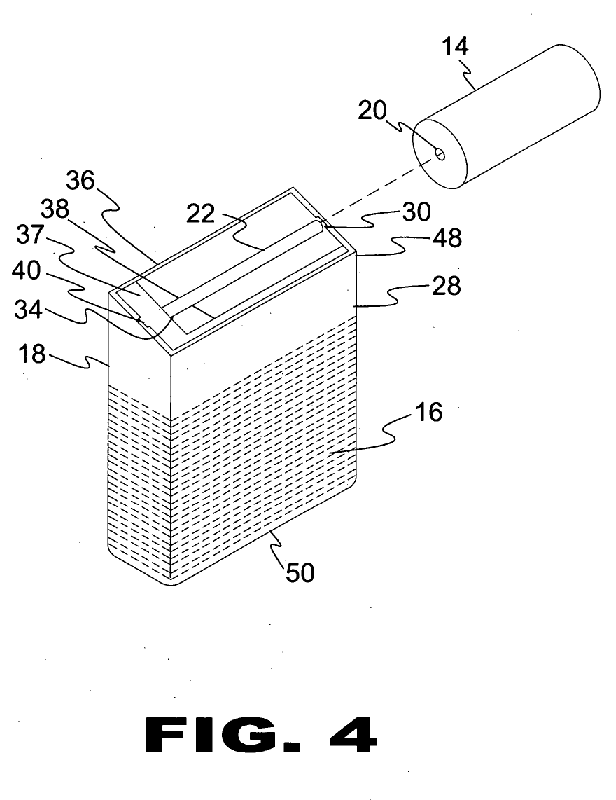

Shown herein, the pin 22 is connected to the housing 18 via a hinge 40, as will be further discussed with reference to FIG. 4. The unhinged end of the pin 22 has been unseated from within its respective channel 30. Removal of the unhinged end of the pin 22 from its channel 30 results in the applicator 14 being pivoted about the hinge 40 and out of its position covering and sealing the reservoir 28 thereby allowing a user to at least one of add more powder to the dispenser reservoir 28 and clean and/or replace the applicator 14. After the user has performed the desired action, the unhinged end of the pin 22 is re-seated and secured within the channel 30 for normal use of the dispenser 10. The pin 22 is secured within the channel 30 via at least one of a snap-fit mechanism and a spring-loading mechanism. The embodiment shown for providing access to the reservoir 28 and releasing applicator 14 are for purposes of example. Alternatively, any means for releasably securing the pin within the channels 30 and the applicator 14 from its position sealing the reservoir 28 may be used. For example, if the pin 22 is integrally formed with the sleeve 20, the pin may be releasably securing in each channel 30 using a snap-fit mechanism or a spring loading mechanism.

FIG. 4 shows an additional aspect of the applicator 14 used with the dispense 10. This embodiment includes the housing 18 having the reservoir 28 with a powder 16 contained therein. The powder 16 is applied to a desired surface via the applicator 14 which is formed from a soft absorbent material 52. The applicator 14 is releaseably and rotatively secured to the top side of the housing 18 by a U-shaped guide 34. The U-shaped guide 34 includes a base member 37. A first guide member 36 and a second guide member 38 extend in parallel outward from respective distal ends of the base member 37. An end of the pin 22 is pivotally connected to an end of the housing 18 via a hinge 40. The pin 22 further extends through substantially a center of the base member 37. The applicator 14 is positioned on the pin 22 so that the pin 22 extends through the sleeve 20 of the applicator 14. Once the pin 22 is inserted through the sleeve 20, the applicator 14 is positioned between the guide members 36, 38. An end of the pin 22 opposite the hinge 40 is then snap-fit into the channel 30 thereby releaseably and rotatively securing the applicator 14 in place. Alternatively, the end of the pin 22 opposite the hinge 40 may be spring loaded and secured in the channel 30 of the housing 18. These methods of securing the pin 22 are described for exemplary purposes only and any way to secure a pin 22 within a channel may be used. When the applicator 14 is secured in place, a seal is formed between the U-shaped guide 34 and the applicator 14 securing the powder 16 contained in the reservoir 28. Alternatively, the base member 37 can be formed without guide members 38,38. In a configuration without guide members 36, 38, the applicator 14 has a diameter such that, upon releasably securing the pin 22 within the channel, a seal is formed between the applicator 14 and the top end 48 of the housing 18.

The embodiment shown in FIG. 4, allows the applicator 14 to be easily removed in order that it may be changed and/or cleaned by the user. This is accomplished by releasing the end of the pin 22 opposite the hinge 40 from the channel 30. The base member 37 is pivoted about the hinge 40 and the applicator and sleeve 20 may be removed from the pin 22. Thereafter, the applicator 14 is slidably removed from the sleeve 20 and can be at least one of cleaned and replaced. This allows the user to utilize the dispenser 10 of the present invention for dispensing a plurality of different types of powder and/or other particulate matter. Additionally, this provides the user with access to the dispenser reservoir 28 to at least one of add additional powder 16 thereto when the supply of powder has been depleted and fill the reservoir with a different type of powder.

This dispenser 10 as described hereinabove with respect to FIGS. 1-4 has been tested using several configurations of the dispenser 10 and a multiplicity of absorbent materials with many different powders as well as with fine dry, very small (300+) mesh materials. During these tests, there was a distinct reduction in the amount of airborne particulates and a substantial savings of wasted powder over conventional shaker can models. Additionally, no ill effects were witnessed by any users of the invention such as eye or lung irritation.

The dispenser 10 according to invention principle as shown in FIGS. 1-4 was tested over a two month period using different types of absorbent material attached to the roller. A first test was performed in order to determine the ability of the dispenser 10 to perform as expected. A dispenser 10 including manufactured sheepskin material used as the soft absorbent material was attached to a roller forming the applicator and used in 571 applications with no failures of powder pick up.

The test was performed using a portable sand blast unit (Photobrasibe Sand Carver 2 with recovery system) with protected hand and arm inserts in a closed vessel having a stagnant air environment. This unit was used to manipulate each of a shaker can and the dispense of the present invention according to their intended modes of use.

A net weight, 10 ounce (283 g) “ordinary” shaker can of Equate Medicated Body Powder was shaken (as in an ordinary from a height of approx five (5) inches) onto a standard cloth, baby diaper. The diaper was folded twice and removed from the closed vessel. Any particulate matter in the air of the closed vessel was allowed to settle for eight (8) hours. Upon expiration of the eight hour time period, the particulate matter residue on the bottom of the closed vessel was slowly and carefully removed using a scraping tool. The amount of settled particulate matter removed was measured to be 20 grams.

The experiment was then repeated in a closed vessel having a stagnant air environment using the dispenser of the present invention. The dispenser was filled with 283 grams of the Equate Medicated Body Powder. The applicator was caused to contact the cloth diaper and the applicator was rotated to apply the powder to the diaper. Upon application, the diaper was folded twice within the vessel and removed therefrom. Any particulate matter in the air was allowed to settle for eight (8) hours. Upon expiration of the eight hour time period, the residue on the bottom of the closed vessel was slowly and carefully removed using a scraping tool. The amount of particulate matter removed was measured to be less than 2 grams.

As a result of the above performed test, the dispenser 10 of the present invention was able to reduce the particulate matter introduced into the air by substantially 90%. Additionally, due to inconsistent edges of the manufactured sheepskin used in the test, a powder bleed thru was experienced to a noticeable extent leading to more powder escaping to potentially become airborne. Despite the bleed through, the powder was successfully able to be retained by the soft absorbent material and applied to a surface. To remedy the effects caused by the bleed through, the soft absorbent material should include rounded (bound) edges thereby lowering the tolerance for bleed through. Using a soft absorbent material having rounded edges may allow for reduction by 97% of particulate matter introduced into the air during ordinary usage.

Therefore, the powder dispenser 10 of the present invention can be used for a myriad of applications. The powder dispenser 10 can be used to apply powder to the underarm area of a person after a shower. Additionally, the dispenser allows a user to apply powder to either their person or to another person and/or animal. The dispenser 10 is able to regulate the amount of powder 16 removed from the reservoir 28 thereby prevents forming a cloud of particulate matter typically associated with ceonventional dry material dispensers. The dispenser 10 is thus also able to minimize the amount of powder dispensed as excess powder 16 as the seal formed between the applicator 14 and the top end 48 will prevent any powder 16 not securely received by the applicator 14 from exiting the reservoir 28. The dispenser 10 is particularly useful in applying powder to a body part of an infant without exposing the infant to potential harmful effects associated with introducing the powder into the lungs of the infant.

Additionally, the dispenser 10 can also be used to apply cosmetics, such as powder eye shadow to an eyelid. Additionally a large embodiment of the dispenser 10 can be used to apply a powdered carpet cleaning product to a carpet. Use of the dispenser 10 in this type of situation decreases the likelihood that the particulate matter will cause ocular and respiratory irritation to either pets or to the person cleaning the carpet. Another use of a large embodiment of the dispenser 10 is to mark off a section of lawn for landscaping purposes.

While certain novel features of this invention have been shown and described and are pointed out in the annexed claims, it is not intended to be limited to the details above, since it will be understood that various omissions, modifications, substitutions and changes in the forms and details of the device illustrated and in its operation can be made by those skilled in the art without departing in any way from the spirit of the present invention.

What is claimed is:

1. An apparatus for dispensing dry lightweight substance comprising:
a housing having a first closed end, and a wall extending from a periphery thereof forming a reservoir for retaining the dry lightweight substance; and
an applicator releasably, rotatably connected to and sealing an end of said reservoir opposite said first closed end so as to seal the substance within said reservoir for selectively retaining the dry lightweight substance within said reservoir, said applicator being partially recessed within said reservoir and including a soft absorbent material extending therearound, wherein upon said apparatus being moved such that said substance contacts said soft absorbent material, an amount of said substance is caused to be retained by said soft absorbent material whereby, placing said applicator against a surface and moving said apparatus along the surface causes said applicator to rotate thereby applying said substance retained by said soft absorbent material to the surface.

2. The apparatus as recited in claim 1, wherein said soft absorbent material is at least one of a natural and synthetic material.

3. The apparatus as recited in claim 2 wherein said natural material is at least one of sheepskin, sponge, organically produced foam, cotton and animal skin having short tightly woven hairs thereon.

4. The apparatus as recited in claim 2, wherein said synthetic material is at least one of nylon, acrylic, porous (dry form) foam, plastic foam, and chemically produced foam.

5. The apparatus as recited in claim 1, wherein said applicator is releaseably connected to said housing allowing said applicator to be at least one of cleaned and replaced.

6. The apparatus as recited in claim 1, wherein the dry lightweight substance is a material having a mesh count of substantially 300 mesh.

7. The apparatus as recited in claim 1, wherein upon application via said applicator, said dispenser at least one of minimizes and eliminates an amount of dry lightweight substance from becoming airborne.

8. The apparatus as recited in claim 1, wherein said apparatus reduces irritation of at least one of eyes, nose, throat and lungs associated with ingestion of airborne dry lightweight substance.

9. The apparatus as recited in claim 1, wherein said applicator comprises:
a sleeve having a channel extending through a central section thereof and along a length thereof for positioning said soft absorbent material therearound; and
a pin for receipt within said channel of sleeve and for connecting said applicator to said housing, wherein said soft absorbent material and said sleeve are rotatable 360 degrees about said pin.

10. The apparatus as recited in claim 9, wherein said pin is formed integral with said sleeve.

11. The apparatus as recited in claim 9, wherein said soft absorbent materials extends around an entire face of said sleeve.

12. The apparatus as recited in claim 9, wherein said soft absorbent material is formed integral with said sleeve.

13. The apparatus as recited in claim 9, wherein said soft absorbent material includes a first edge and a second edge opposite and parallel to said first edge and further comprising a securing mechanism for releaseably securing said first edge to said second edge and further around said sleeve.

14. The apparatus as recited in claim 13, wherein said securing mechanism is at least one of a snap, hook and loop tape and a button.

15. The apparatus as recited in claim 1, wherein said housing further comprising
two channels formed in either side of said wall of said housing opposite said first closed end and aligned substantially 180 degrees from each other, and
a locking mechanism for receipt within each of said channels for releaseably securing said applicator to said housing.

16. The apparatus as recited in claim 15, wherein said locking mechanism includes a pin extending through a central section of said applicator along a length thereof whereby said applicator is rotatable 360 degrees about said pin.

17. The apparatus as recited in claim 16, wherein said pin is releaseably secured within said channel by at least one of a snap-fit mechanism and a tension spring mechanism.

18. The apparatus as recited in claim 1, said housing includes
a channel formed in a first side of said wall opposite said first closed end for releaseably securing said applicator to housing, and
a hinge connected to each of said applicator and to a second side of said wall opposite said first side and parallel to said channel, wherein said hinge and said channel are aligned substantially 180 degrees from one another, whereby upon releasing said applicator from within said channel, said applicator is selectively pivotable about said hinge thereby providing access to said reservoir.

19. The apparatus as recited in claim 18, wherein said applicator further comprises a pin releaseably extending through a central section thereof and along a length thereof, wherein a first distal end of said pin is connected to said hinge and a second distal end is releaseably secured within said channel.

20. The apparatus as recited within claim 1, further comprising a guide member for releaseably connecting said applicator to said housing and for sealing said substance within said reservoir.

21. The apparatus as recited in claim 20, wherein said guide member comprises
a base member,
two vertical support members extending outward from distal ends of said base member,
a pin extending through a central section of said base member for receiving said applicator thereon for releasably securing said applicator to said housing and for connecting said applicator to said wall of said housing via a hinge, wherein upon said applicator is positioned on said pin between said vertical support members and releaseably securing said applicator to said housing, said vertical support members and said applicator seal said substance within said reservoir.

22. The apparatus as recited in claim 1, further comprising a cap releaseably connected to said housing for covering said soft absorbent material of said applicator.

23. The apparatus as recited in claim 1, wherein all edges of said soft absorbent material are at least one of rounded and bound.

24. A method for applying a dry light weight substance comprising the activities of:
filling a reservoir of a dispenser with the dry lightweight substance;
sealing the substance within the reservoir of the dispenser using an applicator formed from a soft absorbent material;
moving the dispenser such that the substance in the reservoir is caused to contact the soft absorbent material;
retaining the substance by the soft absorbent material;
contacting the applicator to a desired surface to which the substance is to be applied;
moving the dispenser along the desired surface causing the applicator to rotate; and
depositing the substrate retained by the soft absorbent material on the desired surface.
